(19) United States
(12) Patent Application Publication
(10) Pub. No.: US 2009/0192520 A1
(43) Pub. Date: Jul. 30, 2009

(54) TOOL HOLDER

(75) Inventor: Patrick Armstrong FINLAY, Beaconsfield (GB)

Correspondence Address: EGBERT LAW OFFICES, 412 MAIN STREET, 7TH FLOOR, HOUSTON, TX, 77002, US

(73) Assignee: PROSURGICS Ltd, High Wycombe (GB)

(21) Appl. No.: 12/355,960

(22) Filed: Jan. 19, 2009

(30) Foreign Application Priority Data
Jan. 25, 2008 (GB) 0801418.5

(51) Int. Cl. A61B 19/00; A47F 5/00; B29C 65/00

(52) U.S. Cl. 606/130; 248/342; 156/145

(57) ABSTRACT

A surgical tool support device includes an arm. The arm includes a flexible tubular main body; and a plurality of beads contained within the tubular main body. In a first configuration, a fluid at least partially surrounds the plurality of beads. In a second configuration, at least some of the fluid is evacuated such that the rigidity of the arm is increased.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The present invention relates to a surgical tool supporting device, a method of manufacturing a surgical tool supporting device, a valve module for coupling to a surgical tool supporting device, a support arm, and a surgical system.

2. Description of Related Art Including Information Disclosed Under 37 CFR 1.97 and 37 CFR 1.98.

Endoscopic surgery is known. It is also known to provide a robot, which is configured to hold and maneuver an endoscope during endoscopic surgery.

During such surgical operations it is often necessary for a tool to be held in a substantially permanent position. Such a tool may, for example, comprise a retractor, which is inserted through an incision in a patient and utilized to hold a part of an internal organ of the patient away from the area, which is the subject of the operation. One such example of the use of a retractor in this manner is the retraction of a lobe of a liver in bariatric surgery. This role is normally performed by an assistant surgeon, who holds the tool and performs any minor adjustments in the position of the tool, which may be required in order to assist the surgeon.

This approach has drawbacks. That is, an assistant surgeon may be required to hold the tool in exactly the same location for several hours. As such, the assistant may not hold the tool in a sufficiently static position, the assistant may make an undesired movement of the tool due to miscommunication between the assistant and the surgeon, or the assistant may undesirably occupy valuable space within the operating theater.

### BRIEF SUMMARY OF THE INVENTION

The present invention seeks to ameliorate the problems associated with the prior art.

Accordingly, one aspect of the present invention provides a support arm comprising: a flexible tubular main body; and a plurality of beads contained within the tubular main body. In a first configuration, a fluid at least partially surrounds the plurality of beads. In a second configuration, at least some of the fluid is evacuated such that the rigidity of the arm is increased, and the tubular main body is not substantially fluid-tight such that it is necessary to evacuate at least some of the fluid in the arm throughout a period in which the arm is in the second configuration.

Preferably, the plurality of beads is all of uniform size and shape.

Alternatively, the plurality of beads includes beads of differing sizes.

Preferably, the plurality of beads includes beads of differing shapes.

Advantageously, the beads are manufactured from polystyrene.

Conveniently, opposing parts of the tubular main body across a diameter thereof are resiliently biased apart.

Alternatively, opposing parts of the tubular main body across a diameter thereof are resiliently biased together.

Preferably, the arm is adapted to support more than one type of tool.

Conveniently, the arm is adapted to support more than one tool.

Another aspect of the present invention provides a surgical tool support device including an arm according to the above.

Preferably, the device further comprises a tool clamp configured for attachment to the arm and adapted to hold a tool.

Conveniently, the device further comprises a valve module adapted to control the evacuation of fluid from the flexible tubular main body.

Advantageously, the device further comprises a surgical tool supported by the arm.

Preferably, the surgical tool is a retractor.

Another aspect of the present invention provides a surgical system including a surgical robot and a surgical tool support device according to the above.

Preferably, the surgical robot and surgical tool support device are attached to each other.

Advantageously, the surgical robot is configured to support a first surgical tool, and the tool support device is configured to support a second surgical tool.

Another aspect of the present invention provides a method of manufacturing a support arm, the method comprising the steps of: forming a flexible tubular main body; and providing a plurality of beads within the tubular main body to form the arm. In a first configuration, a fluid at least partially surrounds the plurality of beads. In a second configuration, at least some of the fluid is evacuated such that the rigidity of the arm is increased, and the tubular main body is not substantially fluid-tight such that it is necessary to evacuate at least some of the fluid in the arm throughout a period in which the arm is in the second configuration.

Preferably, the step of providing a plurality of beads comprises providing a plurality of beads, which are all of uniform size and shape.

Alternatively, the step of providing a plurality of beads comprises providing a plurality of beads including beads of differing sizes.

Preferably, the step of providing a plurality of beads comprises providing a plurality of beads including beads of differing shapes.

Advantageously, the step of providing a plurality of beads comprises providing a plurality of beads, which is manufactured from polystyrene.

Conveniently, forming the tubular main body comprises forming a main body in which opposing parts of the tubular main body across a diameter thereof are resiliently biased apart.

Preferably, forming the tubular main body comprises forming a main body in which opposing parts of the tubular main body across a diameter thereof are resiliently biased together.

Another aspect of the present invention provides a method of manufacturing a surgical tool supporting device including an arm the method comprising manufacturing an arm according to any one of claims.

Preferably, the method further comprises the step of providing a tool clamp configured for attachment to the arm and adapted to hold a tool.

Advantageously, the method further comprises the step of providing a valve module adapted to control the evacuation of fluid from the flexible tubular main body.

### BRIEF DESCRIPTION OF THE SEVERAL VIEWS OF THE DRAWINGS

An embodiment of the present invention will now be described, by way of example, with reference to the accompanying drawings.

FIG. 1 shows a sectional schematic view of an arm in accordance with an embodiment of the present invention in a first configuration.

FIG. 2 shows a sectional schematic view of an arm in accordance with an embodiment of the present invention in a second configuration.

FIG. 3 shows a schematic view of an arm in accordance with an embodiment of the present invention being utilized in a surgical operation.

FIG. 4 shows a schematic view of an arm in accordance with an embodiment of the present invention being utilized in a surgical operation in combination with a robot.

### DETAILED DESCRIPTION OF THE INVENTION

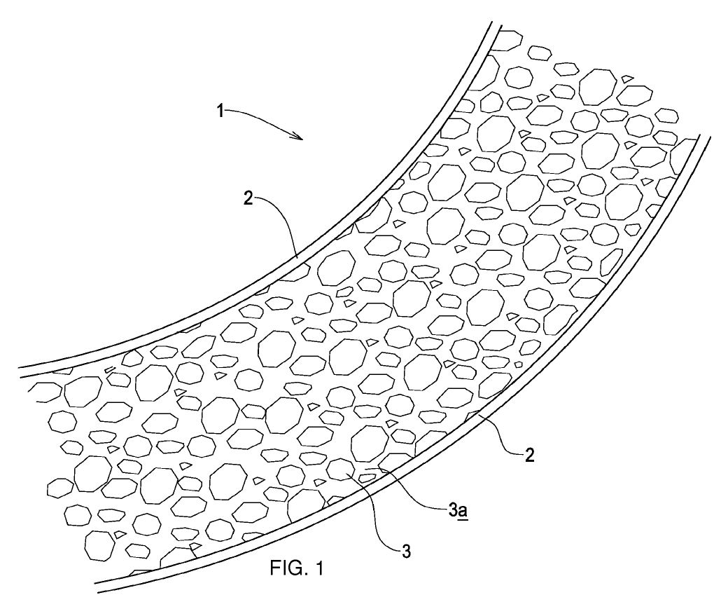

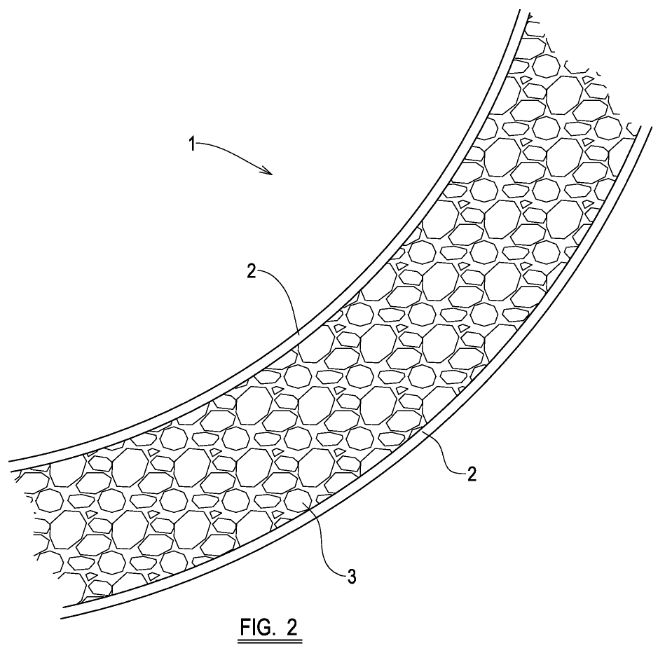

An arm 1 of a surgical tool support device in accordance with an embodiment of the present invention comprises a tubular elongate body having an outer wall 2 defining an inner cavity 3a (see FIGS. 1 and 2).

The tubular elongate body of the arm 1 may have a circular cross-section or may have a different shaped cross-section (e.g. triangular or hexagonal).

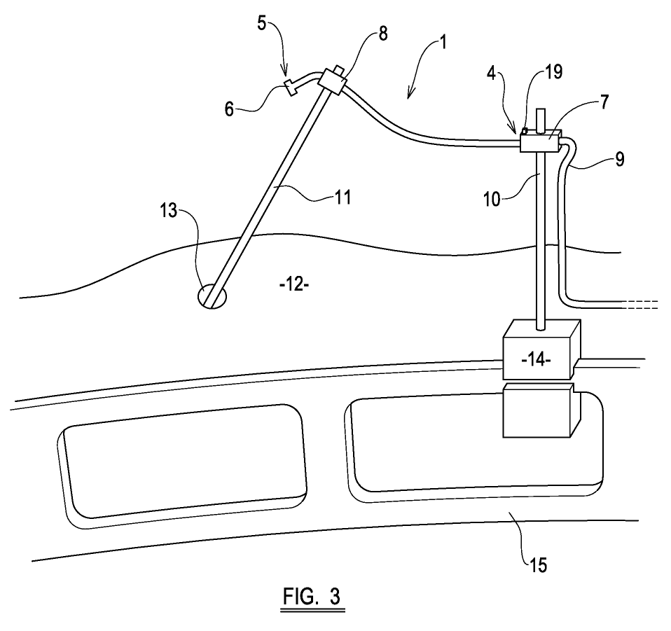

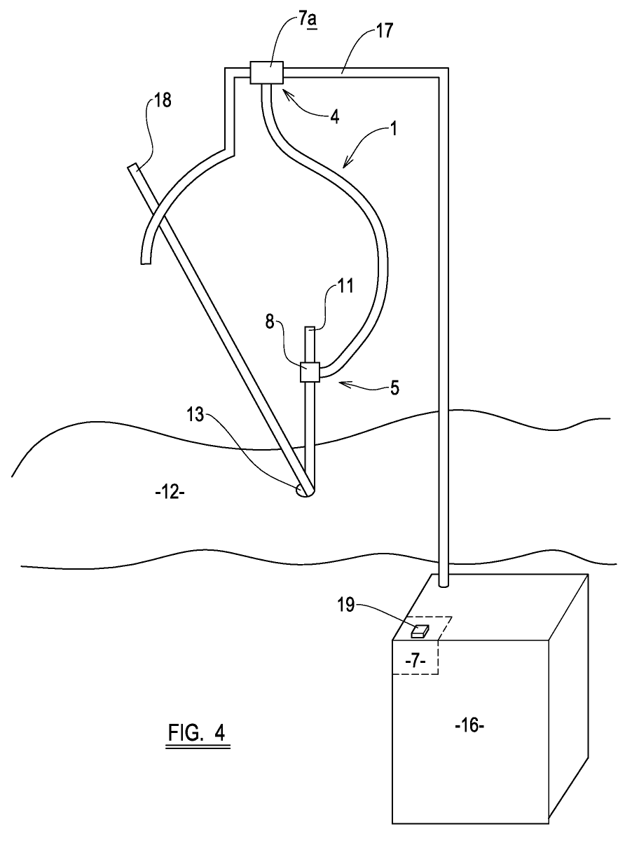

A distal end 5 (see FIGS. 3 and 4) of the arm 1 is substantially sealed. This may be achieved by the use of a plug 6 (as shown in FIG. 3). The plug 6 may be a separate part, which is inserted into the inner cavity 3a of the tubular elongate body of the arm 1 at the distal end 5 thereof. The plug 6 may have a friction fitting, may be adhered to the outer wall 2 of the arm 1, or may be integral with the outer wall 2.

Alternatively, the distal end 5 of the arm 1 may be substantially sealed by adherence of opposing parts of the outer wall 2 of the arm 1 together. This may be achieved by using a heated clamp to melt or partially melt the outer wall 2 of the arm 1 such that when the heated clamp is removed opposing parts of the outer wall are adhered to each other.

The outer wall 2 of the arm 1 is constructed from a flexible material, which is preferably a polymer material. Conveniently, the outer wall 2 is manufactured out of a material, which is substantially fluid-tight (e.g. airtight or watertight).

In an embodiment, the outer wall 2 of the arm 1 is resiliently biased into an open configuration. In an embodiment, the outer wall 2 of the arm 1 includes a biasing element (not shown) which biases opposing parts of the outer wall 2 apart (in an open configuration) in order to maximize the size of the inner cavity 3a (for example, a helical spring of resilient material may be embedded in the outer wall 2, or one or more wall separating members may be located in the inner cavity 3a of the arm 1 and configured to bias opposing parts of the outer wall 2 apart).

A proximal end 4 (see FIGS. 3 and 4) of the arm 1 opposes the distal end 5 of the arm 1 across the length of the arm 1. The proximal end 4 of the arm 1 is coupled to a valve module 7. The valve module 7 has a fluid input (not shown) and a fluid output (not shown), which are generally in fluid communication with each other. A valve is located between the fluid input and the fluid output and is configured to control the flow of fluid through the valve module 7 between the fluid input and output thereof. Preferably, the valve is user actuated by, for example, a control switch 19. The control switch 19 may, in fact, be a push-button switch, which is directly actuated by the user or may be a switch, which is computer controlled.

In an embodiment, the valve may be moved between a first configuration in which the flow of fluid between the input and output is substantially unhindered and a second configuration in which the flow of fluid between the input and output is substantially prevented. Preferably, the valve can be moved to any one of a plurality of positions between the aforementioned first and second configurations.

The proximal end 4 of the arm 1 is coupled to the fluid input of the valve module 7 and may be substantially sealed thereto (such that little or no fluid in the cavity 4 of the arm 1 can escape from the arm 1 except through the fluid input of the valve module 7). In an embodiment, a filter member (not shown) is secured across the entire (or substantially the entire) cross-section of the inner cavity 3a of the arm 1 at the proximal end 4 thereof. The filter member is configured to prevent, substantially, the beads 3 from escaping from the arm 1 but to allow the flow of fluid into and out of the inner cavity 3a of the arm 1. The filter member may form part of the valve module 7.

The fluid output of the valve module 7 is configured to be able to be coupled to a fluid escape conduit 9. This conduit 9 may, for example, be a tube, which is attached to a pump or compressor such that the fluid pressure in the tube is less than that of the surrounding atmosphere and/or less than the fluid pressure in the cavity 4 of the arm 1. In an embodiment, the conduit 9 is a vacuum line such as may be found in an operating theatre or laboratory. In an embodiment, the conduit 9 forms part of a support structure 17 for the arm 1 (as shown in FIG. 4) and the valve module 7 may be located remote from the arm 1 (and may form part of a robot 16 as shown in FIG. 4).

A second fluid input (not shown) may be provided in the valve module 7. This second fluid input may be in fluid communication with the fluid input of the module 7 (hereinafter, the “first fluid input” for clarity). The valve of the valve module 7 (hereinafter, the “first valve” for clarity) or a second valve of the module 7 may be configured to control the flow of fluid between the first fluid input and the second fluid input in a substantially similar manner to the control over the flow of fluid between the first fluid input and the fluid output.

The second fluid input may be configured to be connected to a source of pressurized fluid or may be vented to the atmosphere.

The second valve may be user actuatable in the same manner as the first valve. A separate control switch (not shown) may be provided for this purpose or the control switch 19 maybe adapted for this purpose.

The valve module 7 is adapted so that it may be clamped to a further object. That further object may, for example, be a supporting arm 17 of a robot 16 (as shown in FIG. 4) or may be a support arm 10, which is secured to a frame 15 by a clamp 14 (as shown in FIG. 3). In an embodiment, the valve module 7 is adapted to be clamped to one of a plurality of locations along the further object.

A plurality of beads 3 is located in the inner cavity 3a of the arm 1. There is preferably a sufficiently high density of beads 3 in the inner cavity 3a such that in a first configuration (as will be described in more detail below), the arm 1 may be flexed into a desired shape. However, when in a second configuration, the arm 1 is substantially rigid.

The beads 3 maybe of uniform size and shape; however, the beads 3 are preferably of varying sizes. The shape of the beads 3 may also vary. For example, the beads may have a circular, triangular, hexagonal, or pentagonal cross-section (or, indeed, many other cross-sectional shapes—as will be appreciated). The beads 3 are preferably manufactured from polystyrene. In an embodiment, each bead 3 in an arm 1 is constructed from one of several different materials.

A tool clamp 8 may be secured to the arm 1. In an embodiment, the tool clamp 8 may be secured at any one of a plurality of locations along the length of the arm 1 (as shown in FIG. 3). In an alternative embodiment, the tool clamp 8 is at a fixed location along a length of the arm 1. This fixed location may be the distal end 4 of the arm 1. Indeed, the tool clamp 8 and the plug 6 may be a single unit.

The tool clamp 8 is configured to secure a tool 11 to the arm 1. The tool 11 may have a maximum cross-sectional diameter of 3 to 12mm and the clamp 8 may be adjustable to hold a tool 11 of this diameter.

In preferred embodiments, the inner cavity 3a of the arm 1 is not substantially fluid-tight. This may be achieved by providing a fluid inlet may be provided in the tool clamp 8 or plug 6. The inlet may be a hole (or plurality of holes)—not shown—in the outer wall 2 of the arm 1. The hole (or plurality of holes) may have a smaller largest diameter than any of the beads 3 (in order to prevent their escape) or may be covered (or substantially covered) by a filter for this purpose.

The operation of embodiments of the present invention will now be described.

The basic operation and purpose of an arm 1 in accordance with an embodiment of the present invention is to provide an arm 1 which can be shaped (i.e. flexed) to a desired configuration so as to hold a tool 11 in a substantially fixed location.

This is achieved by providing the arm 1 in a first configuration (as shown in FIG. 1) in which the beads 3 are not tightly packed within the inner cavity 3 a of the arm 1 and the volume around the beads 3 contains a fluid. The arm 1 can then be flexed into the desired configuration. Subsequently, a quantity of the fluid in the inner cavity 3a of the arm 1 is removed (and the arm 1 adopts the second configuration shown in FIG. 2). This results in the outer wall 2 of the arm 1 reducing in diameter. As the number of beads 3 in the arm 1 has not decreased, the beads 3 are now more closely (i.e. densely) packed; this is the second configuration depicted in FIG. 2. In this configuration, the arm 1 is significantly more rigid than when in the first configuration.

More specifically, an arm 1 in accordance with an embodiment of the present invention may be provided. The arm 1 may be supplied to the user in a blister pack or other sterile packaging. The arm 1 is preferably a single use item and a fresh arm 1 is utilized for each surgical operation in which such an arm 1 is required.

The arm 1 may be configured such that it may be attached to a valve module 7 at a proximal end 4 of the arm 1 (as shown in FIG. 3). The arm 1 may also be configured to attach to an attachment arrangement 7a that forms part of a support structure 17 or robot 16, with the valve module 7 remote from the attachment arrangement 7a (as shown in FIG. 4). In an embodiment, the arm 1 is supplied with a valve module 7 already attached to the proximal end 4 thereof.

A tool 11 is attached to the arm 1 by the use of a tool clamp 8, which may be located at the distal end 5 of the arm 1 (as shown in FIG. 4). Alternatively, the tool clamp 8 may be configured to attach to the arm 1 at any one of a plurality of locations along its length (as shown in FIG. 3).

The tool 11 is positioned in the desired location causing the arm 1 to adopt the desired shape and position. A user may then actuate the valve module 7, preferably by depression of the control switch 19, to cause fluid within the arm to be removed from the hollow cavity 4 of the arm 1. The fluid passes through the fluid input and fluid output of the valve module 7 and is drawn along the fluid escape conduit 9 (due to the lower fluid pressure in the conduit 9 compared to the fluid pressure in the inner cavity 3a of the arm 1). This causes the outer wall 2 of the arm 1 to collapse at least partially to reduce the volume of the inner cavity 3a. As the number of beads 3 within the inner cavity 3a is not substantially reduced, the arm 1 becomes more rigid. If sufficient rigidity is obtained then the tool 11 is substantially held in fixed position.

If the inner cavity 3a of the arm 1 is sufficiently fluid-tight (apart from the fluid inputs and outputs described above), then there may not be a need to evacuate at least part of the fluid in the arm 1 continuously when the arm 1 is in the second configuration. However, it is envisaged that the inner cavity 3a may not be substantially fluid-tight and that, therefore, in order to maintain the desired degree of rigidity of the arm 1 it is necessary to evacuate at least some of the fluid in the arm 1 throughout the period during which the arm 1 is in the second configuration; this may be done continuously or periodically.

If movement of the tool 11 and the arm 1 is required during an operation, then the valve module 7 is actuated to allow fluid to pass into the inner cavity 3a of the arm 1 from the second fluid input to the first fluid input (which now acts as a fluid output). If the inner cavity 3a of the arm 1 is not fluid-tight, then in order to increase the volume of fluid in the inner cavity 3a of the arm 1 it may only be necessary to discontinue any previous continuous or periodic evacuation of the arm 1, in order to allow fluid to enter the inner cavity 3a of the arm 1.

The entry of additional fluid into the inner cavity 3a of the arm 1 causes the volume thereof to increase at least partially. This process reduces the rigidity of the arm 1 and allows the arm 1 to be repositioned. The fluid in the inner cavity 3a can subsequently be at least partially removed (as described above) to increase the rigidity of the arm 1 once again.

The fluid in the arm 1 maybe partially or fully evacuated in the above-described embodiment to increase the rigidity of the arm 1. Similarly, the quantity of fluid, which may be passed back into the arm 1 can, in some embodiments, be controlled to decrease the rigidity of the arm 1. The introduction and evacuation of fluid from the arm 1 may be a continuous-type process by adjustment of the valves of the valve module 7 (in which the quantity of fluid can be varied according to a plurality of different levels between substantially fully evacuated and substantially full inflated) or may be discrete-type process (in which the arm 1 is either substantially fully evacuated or substantially fully inflated)—in other words, the valves of the valve module 7 are either in an open or closed position only.

The fluid may be air, nitrogen, water, saline solution, a gel (i.e. a liquid with a higher viscosity than water), or any other fluid, which is available to the skilled person and achieves the desired result. It is preferable to use a non-toxic fluid, which is also preferably sterile (or substantially sterile) such that, in the event of a leak of the fluid during use, the health of the patient (and surgeons etc) is not put at risk. Preferably, the fluid is air.

The arm 1 may be manufactured in a number of different manners. For example, a polymer tube may be extruded and segmented into lengths (each length will ultimately form an arm 1). Each length of the extruded polymer tube may be clamped in a heated clamp at one end thereof to form a seal. From the open end of the tube a predetermined quantity of beads may be inserted in to an inner cavity defined by the outer wall of the extruded tube. A filter member may then be attached to the open end of the tube. Thus, an arm 1 is formed. In an embodiment, the valve module 7 is attached to the open end of the tube at this stage.

Each arm 1 maybe 35 cm in length.

It will be appreciated that the arm 1 maybe adapted to support more than one tool and/or more than one type of tool. In an embodiment, the arm 1 terminates in a tool holder. Preferably the tool holder can hold at least two different types of tool. Advantageously the tool holder can hold at least two tools at a time.

Other manufacturing methods will be understood by the person skilled in the art to be equally applicable.

Embodiments of the present invention, therefore, provide a simple and inexpensive supporting arm 1 for use in a surgical operation and of particular use in endoscopic surgical operations. There are no complex arrangements of moving parts to allow the arm 1 to lock in position. Thus, the arm 1 can be produced for single use. This avoids the need to sterilize the arm 1 between operations or to drape the arm 1 during operations.

It is not necessary for the inner cavity 3 a of the arm 1 to be entirely fluid-tight as a continuous or periodic evacuation of fluid from the arm 1 can be utilized to maintain the rigidity of the arm 1. Thus, expensive sealing and other manufacturing techniques are not required.

Once the arm 1 has been utilized and disposed of, the arm 1 may be at least partially recycled by removal of the beads 3. The beads 3 may be sterilized and reused in a new arm 1 or may be, if made of an appropriate material, melted and molded into a different product. The polymer tube forming the outer wall 2 of the arm may be melted down and molded into a different product (if made of an appropriate material).

It will be appreciated that the arm 1 has been described above with the normal state of the arm 1 being flexible (i.e. there is sufficient fluid in the arm 1 to allow the arm to be positioned in the desired configuration) and opposing parts of the outer wall 2 being biased apart. However, this is not essential. Indeed, the normal state of the arm 1 may be a rigid state with any fluid in the arm 1 being substantially evacuated. Opposing parts of the outer wall 2 may additionally be resiliently biased towards each other. Thus, in order to change the state of the arm 1 to a flexible configuration it is necessary to introduce fluid into the inner cavity 3a of the arm 1. Once the arm 1 has been correctly positioned, the fluid can be evacuated. As such, the fluid evacuation conduit 9, described above, may be replaced by a fluid supply conduit 9.

In an embodiment, the tool clamp 8 is omitted and the distal end 5 of the arm 1 is, itself, utilized as a tool. To this end, the distal end 5 of the arm 1 may be shaped to form a support surface adapted to retract the whole or part of an internal organ of a patient.

Embodiments of the present invention may be used in combination with a surgical robot 16 (As shown in FIG. 4). The surgical robot 16 may be configured to hold and/or move a surgical tool 18 which may be, for example, an endoscope or retractor.

Embodiments of the present invention may be utilized in a method for performing surgery comprising the steps of: providing a surgical tool support device including an arm comprising a tubular main body containing a plurality of beads and a fluid which at least partially surrounds the plurality of beads; attaching a surgical tool to the device; positioning the surgical tool by flexing the tubular main body; evacuating a fluid from at least part of the tubular main body such that the rigidity of the arm is increased.

A method as described above may be performed wherein the step of positioning the surgical tool includes the step of position a surgical retractor to retract part of a patient's anatomy.

It will be understood that the embodiments described above are just an example of an implementation of the present invention. It is envisaged that embodiments of the invention may be utilized in non-surgical applications.

When used in this specification and claims, the terms, “comprises” and “comprising” and variations thereof mean that the specified features, steps or integers are included. The terms are not to be interpreted to exclude the presence of other features, steps or components.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilized for realizing the invention in diverse forms thereof.

1. A support arm comprising:
a flexible tubular main body; and
a plurality of beads contained within the tubular main body,
wherein, in a first configuration, a fluid at least partially surrounds the plurality of beads, and
wherein, in a second configuration, at least some of the fluid is evacuated such that the rigidity of the arm is increased, and the tubular main body is not substantially fluid-tight such that it is necessary to evacuate at least some of the fluid in the arm throughout a period in which the arm is in the second configuration.

2. The arm according to claim 1, wherein the plurality of beads is all of uniform size and shape.

3. The arm according to claim 1, wherein the plurality of beads includes beads of differing sizes.

4. The arm according to claim 1, wherein the plurality of beads includes beads of differing shapes.

5. The arm according to claim 1, wherein the beads are manufactured from polystyrene.

6. The arm according to claim 1, wherein opposing parts of the tubular main body across a diameter thereof are resiliently biased apart.

7. The arm according to claim 1, wherein opposing parts of the tubular main body across a diameter thereof are resiliently biased together.

8. The arm according to claim 1, adapted to support more than one type of tool.

9. The arm according to claim 1, adapted to support more than one tool.

10. A surgical tool support device comprising:
an arm according to claim 1.

11. The device according to claim 10, further comprising a tool clamp configured for attachment to the arm and adapted to hold a tool.

12. The device according to claim 10, further comprising a valve module adapted to control the evacuation of fluid from the flexible tubular main body.

13. The device according to claim 10, further comprising a surgical tool supported by the arm.

14. The device according to claim 13, wherein the surgical tool is a retractor.

15. A surgical system comprising:
a surgical robot; and
a surgical tool support device according to claim 10.

16. The system according to claim 15, wherein the surgical robot and surgical tool support device are attached to each other.

17. The system according to claim 15, wherein the surgical robot is configured to support a first surgical tool and the tool support device is configured to support a second surgical tool.

18. A method of manufacturing a support arm, the method comprising the steps of:
forming a flexible tubular main body; and
providing a plurality of beads within the tubular main body to form the arm,
wherein, in a first configuration, a fluid at least partially surrounds the plurality of beads, and
wherein, in a second configuration, at least some of the fluid is evacuated such that the rigidity of the arm is increased, and the tubular main body is not substantially fluid-tight such that it is necessary to evacuate at least some of the fluid in the arm throughout a period in which the arm is in the second configuration.

19. The method according to claim 18, wherein the step of providing a plurality of beads comprises providing a plurality of beads which are all of uniform size and shape.

20. The method according to claim 18, wherein the step of providing a plurality of beads comprises providing a plurality of beads including beads of differing sizes.

21. The method according to claim 18, wherein the step of providing a plurality of beads comprises providing a plurality of beads including beads of differing shapes.

22. The method according to claim 18, wherein the step of providing a plurality of beads comprises providing a plurality of beads which are manufactured from polystyrene.

23. The method according to claim 18, wherein forming the tubular main body comprises forming a main body in which opposing parts of the tubular main body across a diameter thereof are resiliently biased apart.

24. The method according to claim 18, wherein forming the tubular main body comprises forming a main body in which opposing parts of the tubular main body across a diameter thereof are resiliently biased together.

25. A method of manufacturing a surgical tool supporting device, the method comprising the steps of:
manufacturing an arm according to claim 18.

26. The method according to claim 25, further comprising the step of providing a tool clamp configured for attachment to the arm and adapted to hold a tool.

27. The method according to claim 25, further comprising the step of providing a valve module adapted to control the evacuation of fluid from the flexible tubular main body
